(19) United States
(12) Patent Application Publication
(10) Pub. No.: US 2018/0256541 A1
(43) Pub. Date: Sep. 13, 2018

(54) COMPOSITIONS HAVING ANTI-FUGETACTIC PROPERTIES FOR TREATMENT OF CANCER

(71) Applicant: THE GENERAL HOSPITAL CORPORATION DBA MASSACHUSETTS GENERAL HOSPITAL, Boston, MA (US)

(72) Inventor: Mark C. Poznansky, Newton Center, MA (US)

(21) Appl. No.: 15/760,775

(22) PCT Filed: Sep. 16, 2016
(86) PCT No.: PCT/US16/52337

§ 371 Date: Mar. 16, 2018

Related U.S. Application Data
(60) Provisional application No. 62/220,928, filed on Sep. 18, 2015.

(51) Int. Cl. A61K 31/395; A61K 35/17; A61K 35/15; A61P 35/00; C12N 5/0783; C12N 5/0786; A61K 45/06; A61K 39/00; A61K 39/39

(52) U.S. Cl. CPC A61K 31/395; A61K 35/17; A61K 35/15; A61P 35/00; C12N 5/0636; C12N 5/0645; A61K 2039/55511; A61K 39/0011; A61K 39/39; C12N 2501/73; C12N 2501/22; A61K 2039/5154; A61K 2039/575; A61K 45/06

(57) ABSTRACT

This invention provides ex vivo methods for making modified PBMC compositions having overall anti-fugetactic properties for the effective and efficient treatment of tumors or cancers in a patient, and compositions and use thereof, following treatment with an antigen presenting cell-based vaccine against a cancer antigen.

### CROSS-REFERENCE TO RELATED APPLICATIONS

This application claims priority under 35 U.S.C. § 119(e) to U.S. Provisional Application No. 62/220,928, filed Sep. 18, 2015, which is incorporated herein by reference in its entirety.

### BACKGROUND OF THE INVENTION

Cell movement in response to specific stimuli is observed in prokaryotes and eukaryotes. Cell movement in these organisms has been classified into three types: chemotaxis, or the movement of cells along a gradient towards an increasing concentration of a chemical; negative chemotaxis, which has been defined as the movement down a gradient of a chemical stimulus; and chemokinesis, or the increased random movement of cells induced by a chemical agent.

Chemotaxis and chemokinesis occur in mammalian cells in response to a class of proteins, called chemokines. Additionally, chemorepellent, or fugetactic, activity has been observed in mammalian cells. For example, some tumor cells secrete concentrations of chemokines that are sufficient to repel immune cells from the site of a tumor, thereby reducing the immune system's ability to target and eradicate the tumor. Metastasizing cancer cells may use a similar mechanism to evade the immune system. Repulsion of immune cells, such as tumor antigen-specific T-cells, e.g. from a tumor expressing high levels of CXCL12 or interleukin 8 (IL-8), allows the tumor cells to evade immune control.

CXCR4 is a protein that in humans is encoded by the CXCR4 gene. CXCR4 is expressed by multiple normal cells as well as on tumors. CXCR4 is an alpha-chemokine receptor specific for stromal-derived-factor-1 (SDF-1, also known as CXCL12), a molecule endowed with potent chemotactic activity for lymphocytes. As many as 85% of solid tumors and leukemias express CXCL12 at a level sufficient to have fugetactic effects, e.g. repulsion of immune cells from the tumor. Cancers that frequently express CXCL12 at such levels include, but are not limited to, prostate cancer, lung cancer, breast cancer, pancreatic cancer, ovarian cancer, gastric cancer, esophageal cancer, and leukemia.

Anti-fugetactic agents inhibit the fugetactic activity of tumor cells and allow the patient's immune system to target the tumor. Anti-fugetactic agents and the systemic delivery of anti-fugetactic agents are known in the art (see, for example, U.S. Patent Application Publication No. 2008/0300165, incorporated herein by reference in its entirety). However, the delivery of anti-fugetactic agents as heretofore described will likely result in a portion of the anti-fugetactic agent binding to the CXCR4 receptors on a tumor or other site thus making the effective concentration of the anti-fugetactic agent that binds to immune cells unpredictable.

Prostate cancer is the most common non-cutaneous cancer among men in the United States and is the second leading cause of death from cancer in men. Localized prostate cancer may be cured with surgery or radiation therapy, but the disease recurs in approximately to 30% of patients. Sipuleucel-T (commercially available as PROVENGE® suspension for intravenous infusion) is an active cellular immunotherapy consisting of modified autologous peripheral-blood mononuclear cells (PBMCs), including antigen-presenting cells (APCs), that have been activated ex vivo with a recombinant fusion protein (PA2024). PA2024 consists of a prostate antigen, prostatic acid phosphatase (PAP), that is linked to granulocyte-macrophage colony-stimulating factor (GM-CSF), an immune-cell activator. During ex vivo culture with PAP-GM-CSF, the APCs take up and process the recombinant target antigen into small peptides that are then displayed on the APC surface. Following administration to the patient, the modified cells trigger the immune system to produce T-cells that kill any cell having the PAP, namely, prostate cancer cells.

Accordingly, there remains a need for methods and compositions that target tumors and cancers, particularly prostate cancer, to efficiently kill tumors and/or metastasizing cancer cells.

### SUMMARY OF THE INVENTION

It has now been surprisingly discovered that the anti-fugetactic properties imparted by at least some anti-fugetactic agents, such as AMD3100, resides in binding thereof to cell surface receptors, e.g. CXCR4, on the T-cell. Surprisingly, the anti-fugetactic property of these anti-fugetactic agents has been found to be concentration dependent. In particular, it has been discovered that when an immune cell encounters too high a concentration of an anti-fugetactic agent, the anti-fugetactic effect is lost. The immune cell is thus prevented from effectively penetrating a tumor or homing in on a metastasizing cancer cell.

While not being bound by theory, knowing that the CXCR4 receptors have multiple sites in the human body as well as on tumors, and also knowing that the PBMC population in the human body approaches or exceeds one trillion cells, the T-cells that are activated following delivery of Sipuleucel-T are less efficient to effectively eradicate tumors and/or cancer cells in a patient without the presence of an anti-fugetactic agent as described herein.

Based at least in part on the discoveries set forth above, it has been found that the binding of an anti-fugetactic agent to PBMCs, particularly T-cells, or any other immune cells having CXCR4 receptors, ex vivo, provides an improved ability to control the amount of association of the anti-fugetactic agent with the PBMCs (e.g. via CXCR4 or other cell surface receptor that binds the fugetactic agent) to provide a modified PBMC population that, overall, retains the desired anti-fugetactic properties when administered to the patient. That is, the modified PBMC population is able to overcome the fugetactic effect of a tumor or cancer cell in order to effectively target the tumor or cell.

According to the present invention, such modified PBMC populations can be administered via any suitable method. In some embodiments, the modified PBMCs are administered locally to, or adjacent to, a tumor or site(s) or cancer cells. Alternatively, the modified PBMC population may be administered systemically, e.g., by intravenous infusion.

Treatment of the patient with unbound anti-fugetactic agent prior to or concurrently with administration of the modified PBMCs provides further improvements in anti-fugetactic response and tumor targeting of the PBMCs. In particular, it is contemplated that the treatment with unbound anti-fugetactic agent will result in less competition for the anti-fugetactic agent bound to CXCR4 on the infused immune cells. That is, at least a subset of endogenous CXCR4 receptors encountered by the infused cells will be occupied by the anti-fugetactic agent and thus will not be available to compete away anti-fugetactic agent associated with the infused cells.

Similarly, unbound anti-fugetactic agent can be administered via any suitable method, including locally or systemically.

In one embodiment, the invention relates to an ex vivo immune cell composition comprising immune cells (such as PBMCs, T-cells, etc) that are responsive to a tumor antigen, and an anti-fugetactic agent, wherein said modified immune cell composition has anti-fugetactic properties for the effective and efficient treatment of tumors or cancers in a patient. Preferably, the immune cells are autologous (derived from the patient to be treated).

Suitable anti-fugetactic agents include AMD3100 (mozobil/plerixafor) or derivative thereof, KRH-1636, T-20, T-22, T-140, TE-14011, T-14012, TN14003, TAK-779, AK602, SCH-351125, Tannic acid, NSC 651016, thalidomide, GF 109230X, an antibody that interferes with dimerization of a fugetactic chemokine, or an antibody that interferes with dimerization of a receptor for a fugetactic chemokine. In a preferred embodiment, the anti-fugetactic agent is AMD3100.

In some embodiments, the anti-fugetactic agent is associated with one or more receptors on the immune cell surface. In one embodiment,

In some embodiments, the cell population or composition includes anti-fugetactic agent that is not associated with the cells.

In one embodiment, the immune cells are PBMCs. In preferred embodiments, the cancer is prostate cancer.

In related embodiments the immune cells from a patient that are responsive to a tumor antigen are obtained from the patient after treatment with a vaccine or antigen presenting cell that induces an immune response against the tumor antigen, such as Sipuleucel-T.

In preferred embodiments, the immune cells are induced to be responsive to a tumor antigen by ex vivo incubation with a fusion protein. In some embodiments, the fusion protein comprises a tumor antigen portion and an immune signaling factor portion. The tumor antigen portion may comprise any tumor antigen or portion thereof, e.g. prostatic acid phosphatase (PAP). The immune signaling factor portion may be any protein or portion thereof that activates or facilitates maturation of APCs, e.g. GM-CSF.

In an especially preferred embodiment, the fusion protein is PA2024 (Sipuleucel-T, trade name PROVENGE™). PA2024 is described in more detail in U.S. Pat. No. 6,210,662, which is incorporated herein by reference in its entirety.

In some embodiments, the patient is administered an anti-cancer vaccine to promote an immune response prior to removal of PBMCs from the patient.

Related embodiments include a pharmaceutical composition comprising an effective amount of a modified immune cell composition and one or more pharmaceutically acceptable excipients.

In further embodiments, the invention is a method of treating cancer in a patient who has been immunized against a cancer antigen, comprising administration of an effective amount of an anti-fugetactic agent to the patient. The anti-fugetactic agent may be delivered directly to the tumor, or systemic. In related embodiments, the invention includes the method of first immunizing the patient, and then overcoming the fugetactic properties of the cancer.

The invention is also a method of treating cancer in a patient who has been immunized against a cancer antigen, comprising administration of cell composition or a pharmaceutical composition as elsewhere herein.

Tumor antigens are known in the art. For example, and without limitation, tumor antigens contemplated herein include PAP, alphafetoprotein (AFP), Carcinoembryonic antigen (CEA), CA-125, MUC-1, Epithelial tumor antigen (ETA), Tyrosinase, Melanoma-associated antigen (MAGE), abnormal products of ras, p53, α-folate receptor, CAIX, CD19, CD20, CD30, CD33, EGP-2, erb-B2, erb-B 2,3,4, FBP, GD2, GD3, Her2/neu, IL-13R-a2, k-light chain, LeY, MAGE-A1, Mesothelin, and PSMA. See. e.g. Scott et al., Cancer Immunity 2012, 12:14, which is incorporated herein by reference in its entirety.

One embodiment of the invention relates to a method for treating tumors or cancers, particularly prostate cancer, by the systemic administration of a modified PBMC composition according to the present invention to a patient in need thereof.

One embodiment of the invention relates to a method for treating tumors or cancers, particularly prostate cancer, by the local administration of a modified PBMC composition according to the present invention to (e.g., directly to or into), or adjacent to, a tumor or site(s) or cancer cells in a patient in need thereof.

One embodiment of the invention relates to a method for treating tumors or cancers by the systemic administration of a modified PBMC composition according to the present invention to a patient in need thereof.

In one embodiment, the anti-fugetactic agent is AMD3100 (mozobil/plerixafor; chemical name 1,1′-[1,4-phenylenebis(methylene)]bis [1,4,8,11-tetraazacyclotetradecane]), KRH-1636, T-20, T-22, T-140, TE-14011, T-14012, TN14003, TAK-779, AK602, SCH-351125, Tannic acid, NSC 651016, thalidomide, GF 109230X, an antibody that interferes with dimerization of a fugetactic chemokine, or an antibody that interferes with dimerization of the receptor for a fugetactic chemokine.

In one embodiment, the tumor is a solid tumor. In one embodiment, the tumor is a non-solid tumor. In one embodiment, the tumor is a leukemia.

One embodiment of the invention relates to a method of treating cancer in a patient in need thereof, comprising: a) providing immune cells derived from the patient; b) incubating the immune cells with a fusion protein comprising a tumor antigen portion and an immune signaling factor portion for a period of time sufficient for the immune cells to become responsive to the tumor antigen; c) contacting the immune cells with an anti-fugetactic agent; and d) administering the immune cells to the patient.

One embodiment of the invention relates to a method for making an immune cell composition, the method comprising: a) providing an immune cell composition; b) incubating the immune cells with a fusion protein comprising a tumor antigen portion and an immune signaling factor portion for a period of time sufficient for the immune cells to become responsive to the tumor antigen; and c) contacting the immune cells with an anti-fugetactic agent.

### BRIEF DESCRIPTION OF THE FIGURES

FIG. 1 represents the bimodal chemotactic effect of increasing amounts of AMD3100 on human T cells.

FIG. 2 represents the bimodal fugetactic effect of increasing amounts of AMD3100 on human T cells.

### DETAILED DESCRIPTION OF THE INVENTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about” It is also to be understood, although not always explicitly stated, that the reagents described herein are merely examples and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g. horse, cow, pig, goat, sheep). In especially preferred embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, remission of the cancer, inhibition of metastasis of the tumor, reduction or elimination of at least one symptom of the cancer, and the like.

The term “administering” or “administration” of an agent, drug, or a natural killer cell to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

The term “simultaneous” therapeutic use refers to the administration of at least two active ingredients by the same route and at the same time or at substantially the same time.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect. For example, an effective amount of an anti-fugetactic agent may be an amount sufficient to have an anti-fugetactic effect on a cancer cell or tumor (e.g. to attenuate a fugetactic effect from the tumor or cancer cell). The therapeutically effective amount of the agent will vary depending on the tumor being treated and its severity as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

The term “immunize” as used herein refers to strengthening a patient's immune system against a target, e.g. a cancer. Immunization triggers an immune response against the target.

The term “vaccine” refers to a substance that elicits an immune response and also confers protective immunity upon a subject. The term “vaccine” also refers to immunostimulants, i.e. agents that stimulate the immune system.

An “immune response” refers to the reaction of a subject to the presence of an antigen, which may include at least one of the following: making antibodies, developing immunity, developing hypersensitivity to the antigen, and developing tolerance.

The term “kill” with respect to a cell/cell population is directed to include any type of manipulation that will lead to the death of that cell/cell population.

“Antibodies” as used herein include polyclonal, monoclonal, single chain, chimeric, humanized and human antibodies, prepared according to conventional methodology.

“Cytokine” is a generic term for non-antibody, soluble proteins which are released from one cell subpopulation and which act as intercellular mediators, for example, in the generation or regulation of an immune response. See Human Cytokines: Handbook for Basic & Clinical Research (Aggrawal, et al. eds., Blackwell Scientific, Boston, Mass. 1991) (which is hereby incorporated by reference in its entirety for all purposes).

“CXCR4/CXCL12 antagonist” refers to a compound that antagonizes CXCL12 binding to CXCR4 or otherwise reduces the fugetactic effect of CXCL12.

By “fugetactic activity” or “fugetactic effect” it is meant the ability of an agent to repel (or chemorepel) a eukaryotic cell with migratory capacity (i.e., a cell that can move away from a repellant stimulus), as well as the chemorepellant effect of a chemokine secreted by a cell, e.g. a tumor cell. Usually, the fugetactic effect is present in an area around the cell wherein the concentration of the chemokine is sufficient to provide the fugetactic effect.

Some chemokines, including interleukin 8 and CXCL12, may exert fugetactic activity at high concentrations (e.g., over about 100 nM), whereas lower concentrations exhibit no fugetactic effect and may even be chemoattractant.

Accordingly, an agent with fugetactic activity is a “fugetactic agent.” Such activity can be detected using any of a variety of systems well known in the art (see. e.g., U.S. Pat. No. 5,514,555 and U.S. Patent Application Pub. No. 2008/0300165, each of which is incorporated by reference herein in its entirety). A preferred system for use herein is described in U.S. Pat. No. 6,448,054, which is incorporated herein by reference in its entirety.

The term “immune cells” as used herein are cells of hematopoietic origin that are involved in the specific recognition of antigens. Immune cells include antigen presenting cells (APCs), such as dendritic cells or macrophages, B cells, T cells, and the like.

The term “anti-fugetactic effect” refers to the effect of the anti-fugetactic agent to attenuate or eliminate the fugetactic effect of the chemokine.

The terms ‘T cells” or ‘T lymphocytes”, as used herein, are a type of lymphocyte, i.e., a type of white blood cell, that plays a central role in cell-mediated immunity, and can be distinguished from other lymphocytes, such as B cells and natural killer cells (NK cells), by the presence of a T-cell receptor (TCR) on the cell surface. T cells or T lymphocytes include several subsets of T cells, each having a distinct function. The majority of human T cells rearrange their alpha/beta T cell receptors and are termed alpha beta T cells and are part of adaptive immune system. Specialized gamma delta T cells, which comprise a minority of T cells in the human body (more frequent in ruminants), have invariant TCR (with limited diversity), can effectively present antigens to other T cells and are considered to be part of the innate immune system.

The term “T cell receptor” or “TCR” is a complex of integral membrane proteins that participate in the activation of T-cells in response to an antigen. Stimulation of TCR is triggered by MHC (major histocompatibility complex) molecules on cells with the antigen.

Engagement of the TCR initiates positive and negative cascades that ultimately result in cellular proliferation, differentiation, cytokine production, and/or activation-induced cell death. These signaling cascades regulate T-cell development, homeostasis, activation, acquisition of effector's functions and apoptosis.

The term “peripheral blood mononuclear cell” or “PBMC” is any blood cell having a round nucleus (as opposed to a lobed nucleus). For example: a lymphocyte, a monocyte or a macrophage. These blood cells are a critical component in the immune system to fight infection and adapt to intruders. The lymphocyte population consists of T cells (CD4 and CD8 positive ˜75%), B cells and NK cells (˜25% combined).

The term “antigen-presenting cell” or “APC” or “accessory cell” is a cell that displays foreign antigens complexed with major histocompatibility complexes (MHCs) on their surfaces; this process is known as antigen presentation. T-cells may recognize these complexes using their T-cell receptors (TCRs). T cells cannot recognize, and therefore cannot respond to, ‘free’ antigen. T cells can only ‘see’ an antigen that has been processed and presented by cells via carrier molecules like MHC and CD1 molecules. Most cells in the body can present antigen to CD8⁺ T cells via MHC class I molecules and, thus, act as “APCs”; however, the term is often limited to specialized cells that can prime T cells (i.e., activate a T cell that has not been exposed to antigen, termed a naive T cell). These cells, in general, express MHC class II as well as MHC class I molecules, and can stimulate CD4⁺ (“helper”) T cells as well as CD8⁺ (“cytotoxic”) T cells, respectively. After APCs have phagocytosed pathogens, they usually migrate to the vast network of lymph vessels and are carried by lymph flow to the draining lymph nodes. Each lymph node is a collection point where APCs such as dendritic cells (DCs) can interact with T cells. They do this by chemotaxis, which involves interacting with chemokines that are expressed on the surface of cells (e.g., endothelial cells of the high endothelial venules) or have been released as chemical messengers to draw the APCs to the lymph nodes. During the migration, DCs undergo a process of maturation: they lose most of their ability to further engulf pathogens and they develop an increased ability to communicate with T cells. Enzymes within the cell digest the swallowed pathogen into smaller pieces containing epitopes, which are then presented to T cells by the MHC.

The term “CD3” as used herein, also known as ‘cluster of differentiation 3’ is a protein complex and is composed of four distinct chains. In mammals, the complex contains a CD3γ chain, a CD3δ chain, and two CD3ε chains. These chains associate with the T-cell receptor (TCR) and the ζ-chain to generate an activation signal in T lymphocytes. The TCR, ζ-chain, and CD3 molecules together comprise the TCR complex.

The term “PA2024” as used herein refers to prostatic acid phosphatase (PAP), that is linked to granulocyte-macrophage colony-stimulating factor (GM-CSF) to form the fusion protein PAP-GM-CSF.

The term “Sipuleucel-T” as used herein refers to the commercially available product known as PROVENGE® suspension for intravenous infusion) as described in the Highlights of Prescribing Information that is publically available from the U.S. Food and Drug Administration and incorporated herein in its entirety.

The term “autologous” or “autologous cells” as used herein refers to immune cells obtained from, and then administered to the same patient.

The term “anti-cancer therapy” as used herein refers to known cancer treatments, including chemotherapy and radiotherapy, as well as immunotherapy and vaccine therapy.

### Anti-Fugetactic Agents

The anti-fugetactic agent may be any such agent known in the art. In one embodiment, the anti-fugetactic agent is an anti-fugetactic agent as described in U.S. Patent Application Publication No. 2008/0300165, which is hereby incorporated by reference in its entirety. In a preferred embodiment, the anti-fugetactic agent is AMD3100 (mozobil/plerixafor) or a derivative thereof, KRH-1636, T-20, T-22, T-140, TE-14011, T-14012, TN14003, TAK-779, AK602, SCH-351125, Tannic acid, NSC 651016, thalidomide, GF 109230X, an antibody that interferes with dimerization of a fugetactic chemokine, or an antibody that interferes with dimerization of the receptor for a fugetactic chemokine. For example, the antibody may inhibit dimerization of CXCL12, IL-8, CXCR3, or CXCR4. In one embodiment, the anti-fugetactic agent is an antibody that interferes with binding of the chemokine to its receptor. In an especially preferred embodiment, the anti-fugetactic agent is AMD3100.

In one embodiment, the anti-fugetactic agent is an AMD3100 derivative. AMD3100 derivatives include, but are not limited to, those found in U.S. Pat. Nos. 7,935,692 and 5,583,131 (USRE42152), each of which is incorporated herein by reference in its entirety.

Anti-fugetactic agents include any agents that specifically inhibit chemokine and/or chemokine receptor dimerization, thereby blocking the chemorepellent response to a fugetactic agent. Certain chemokines, including IL-8 and CXCL12 can also serve as chemorepellents at high concentrations (e.g., above 100 nM) where much of the chemokine exists as a dimer. Dimerization of the chemokine elicits a differential response in cells, causing dimerization of chemokine receptors, an activity which is interpreted as a chemorepellent signal. Blocking the chemorepellent effect of high concentrations of a chemokine secreted by a tumor can be accomplished, for example, by anti-fugetactic agents which inhibit chemokine dimer formation or chemokine receptor dimer formation. For example, antibodies that target and block chemokine receptor dimerization, for example, by interfering with the dimerization domains or ligand binding can be anti-fugetactic agents. Anti-fugetactic agents that act via other mechanisms of action, e.g. that reduce the amount of fugetactic cytokine secreted by the cells, inhibit dimerization, and/or inhibit binding of the chemokine to a target receptor, are also encompassed by the present invention. Where desired, this effect can be achieved without inhibiting the chemotactic action of monomeric chemokine.

In other embodiments, the anti-fugetactic agent is a CXCR4 antagonist, CXCR3 antagonist, CXCR4/CXCL12 antagonist or selective PKC inhibitor.

The CXCR4 antagonist can be but is not limited to AMD3100, KRH-1636, T-20, T-22, T-140, TE-14011, T-14012, or TN14003, an antibody to CXCR4, or an antibody that interferes with the dimerization of CXCR4. Additional CXCR4 antagonists are described, for example, in U.S. Patent Pub. No. 2014/0219952 and Debnath et al. Theranostics, 2013; 3(1): 47-75, each of which is incorporated herein by reference in its entirety, and include TG-0054 (burixafor), AMD3465, NIBR1816, AMD070, and derivatives thereof.

The CXCR3 antagonist can be but is not limited to TAK-779, AK602, or SCH-351125, or an antibody that interferes with the dimerization of CXCR3.

The CXCR4/CXCL12 antagonist can be but is not limited to Tannic acid, NSC 651016, or an antibody that interferes with the dimerization of CXCR4 and/or CXCL12.

The selective PKC inhibitor can be but is not limited to thalidomide or GF 109230X.

In a preferred embodiment, the anti-fugetactic agent is AMD3100 (plerixafor). AMD3100 is described in U.S. Pat. No. 5,583,131, which is incorporated by reference herein in its entirety.

In one embodiment, the anti-fugetactic agent is coupled with a molecule that allows targeting of a tumor or cancer. In one embodiment, the anti-fugetactic agent is coupled with (e.g., bound to) an antibody specific for the tumor to be targeted. In one embodiment, the anti-fugetactic agent coupled to the molecule that allows targeting of the tumor or cancer.

### Modified Immune Cell Compositions

According to the present invention, a modified autologous PBMC composition having overall anti-fugetactic properties is prepared ex vivo by first extracting or otherwise isolating autologous immune cells, preferably PBMCs, from blood, bone marrow, or other immune cell-containing organs of a patient having a cancerous tumor or other cancer, according to methods known in the art, to provide an autologous PBMC population. For example, such methods include, but are not intended to be limited to apheresis techniques, specifically leukapheresis. Additionally, commercially available kits may be utilized for the extraction of immune cells, e.g. T-cells, such as with EasySep™ Human T Cell Isolation Kit available from STEMCELL™ Technologies, Inc., British Columbia, CANDADA.

The autologous PBMC population is then treated with an anti-fugetactic agent to produce cells having overall anti-fugetactic properties for the effective and efficient treatment of tumors or cancers in said patient, particularly prostate cancer. As would be understood by one skilled in the art, the amount of the anti-fugetactic agent can be determined as described in U.S. Patent Application Publication No. 2008/0300165, which is incorporated herein by reference in its entirety

The modified autologous PBMC composition can then be stored under conditions known in the art for blood products for the subsequent administration to the patient from which the autologous immune cells were derived. In one embodiment, the modified autologous PBMC population can be stored under conditions known in the art for blood products, and then contacted with the anti-fugetactic agent immediately prior to administration thereof to the patient. In another embodiment, the modified autologous PBMC population is contacted with the anti-fugetactic agent immediately prior to administration of the modified immune cell population or composition to the patient

### Dose and Administration

The modified autologous PBMC composition, as described herein, is administered in vivo, to the patient from which the PBMCs were derived, in effective amounts. The effective amount will depend upon the mode of administration, the particular condition being treated and the desired outcome. It will also depend upon the stage of the condition, the age and physical condition of the subject, the nature of concurrent therapy, if any, and like factors well known to the medical practitioner. For therapeutic applications, it is that amount sufficient to achieve a medically desirable result.

Generally, the dose of the modified autologous PBMC composition of the present invention is from about 5 mg/kg body weight per day to about 50 mg/kg per day of anti-fugetactic agent, inclusive of all values and ranges therebetween, including endpoints. In one embodiment, the dose is from about 10 mg/kg to about 50 mg/kg per day. In one embodiment, the dose is from about 10 mg/kg to about 40 mg/kg per day. In one embodiment, the dose is from about 10 mg/kg to about 30 mg/kg per day. In a preferred embodiment, the dose is from about 10 mg/kg to about 20 mg/kg per day. In one embodiment, the dose does not exceed about 50 mg per day.

In one embodiment, the dose of the modified autologous PBMC composition is from about 50 mg/kg per week to about 350 mg/kg per week of the anti-fugetactic agent, inclusive of all values and ranges therebetween, including endpoints. In one embodiment, the dose of the anti-fugetactic agent is about 50 mg/kg per week of the anti-fugetactic agent. In one embodiment, the dose of the modified autologous PBMC composition is about 60 mg/kg per week of the anti-fugetactic agent. In one embodiment, the dose of modified autologous PBMC composition is about 70 mg/kg per week of the anti-fugetactic agent. In one embodiment, the dose of the modified autologous PBMC composition is about 80 mg/kg per week of the anti-fugetactic agent. In one embodiment, the dose of the modified autologous PBMC composition is about 90 mg/kg per week of the anti-fugetactic agent. In one embodiment, the dose of the modified autologous PBMC composition is about 100 mg/kg per week of the anti-fugetactic agent. In one embodiment, the dose of the modified autologous PBMC composition is about 110 mg/kg per week of the anti-fugetactic agent. In one embodiment, the dose of the modified autologous PBMC composition is about 120 mg/kg per week of the anti-fugetactic agent. In one embodiment, the dose of the modified autologous PBMC composition is about 130 mg/kg per week of the anti-fugetactic agent. In one embodiment, the dose of the modified autologous PBMC composition is about 140 mg/kg per week of the anti-fugetactic agent. In one embodiment, the dose of the modified autologous PBMC composition is about 150 mg/kg per week of the anti-fugetactic agent. In one embodiment, the dose of the modified autologous PBMC composition is about 160 mg/kg per week of the anti-fugetactic agent. In one embodiment, the dose of the modified autologous PBMC composition is about 170 mg/kg per week of the anti-fugetactic agent. In one embodiment, the dose of the modified autologous PBMC composition is about 180 mg/kg per week of the anti-fugetactic agent. In one embodiment, the dose of the modified autologous PBMC composition is about 190 mg/kg per week of the anti-fugetactic agent. In one embodiment, the dose of the modified autologous PBMC composition is about 200 mg/kg per week of the anti-fugetactic agent. In one embodiment, the dose of the modified autologous PBMC composition is about 210 mg/kg per week of the anti-fugetactic agent. In one embodiment, the dose of the modified autologous PBMC composition is about 220 mg/kg per week of the anti-fugetactic agent. In one embodiment, the dose of the modified autologous PBMC composition is about 230 mg/kg per week of the anti-fugetactic agent. In one embodiment, the dose of the modified autologous PBMC composition is about 240 mg/kg per week of the anti-fugetactic agent. In one embodiment, the dose of the modified autologous PBMC composition is about 250 mg/kg per week of the anti-fugetactic agent. In one embodiment, the dose of the modified autologous PBMC composition is about 260 mg/kg per week of the anti-fugetactic agent. In one embodiment, the dose of the modified autologous PBMC composition is about 270 mg/kg per week of the anti-fugetactic agent. In one embodiment, the dose of the modified autologous PBMC composition is about 280 mg/kg per week of the anti-fugetactic agent. In one embodiment, the dose of the modified autologous PBMC composition is about 290 mg/kg per week of the anti-fugetactic agent. In one embodiment, the dose of the modified autologous PBMC composition is about 300 mg/kg per week of the anti-fugetactic agent. In one embodiment, the dose of the modified autologous PBMC composition is about 310 mg/kg per week of the anti-fugetactic agent. In one embodiment, the dose of the modified autologous PBMC composition is about 320 mg/kg per week of the anti-fugetactic agent. In one embodiment, the dose of the modified autologous PBMC composition is about 330 mg/kg per week of the anti-fugetactic agent. In one embodiment, the dose of the modified autologous PBMC composition is about 340 mg/kg per week of the anti-fugetactic agent. In one embodiment, the dose of the modified autologous PBMC composition is about 350 mg/kg per week of the anti-fugetactic agent.

In one aspect of the invention, administration of the modified autologous PBMC composition is pulsatile for a period of time sufficient to have an anti-fugetactic effect (e.g. to attenuate the fugetactic effect of the tumor cell). In one embodiment, an amount of modified autologous PBMC composition is administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours. In one embodiment, an amount of modified autologous PBMC composition is administered every 1 day, 2 days, 3 days, 4 days, 5 days, 6 days, 7 days, 8 days, 9 days, or 10 days.

A variety of administration routes are available. The methods of the invention, generally speaking may be practiced using any mode of administration that is medically acceptable, meaning any mode that produces effective levels of the active compounds without causing clinically unacceptable adverse effects.

In one embodiment, the modified autologous PBMC composition is administered parenterally. In one embodiment, the modified autologous PBMC composition is administered via microcatheter into a blood vessel proximal to a tumor. In one embodiment, the modified autologous PBMC composition is administered via microcatheter into a blood vessel within a tumor. In one embodiment, the modified autologous PBMC composition is administered subcutaneously. In one embodiment, the modified autologous PBMC composition is administered intradermally.

In one embodiment, the modified autologous PBMC composition is administered in a continuous manner for a defined period. In another embodiment, modified autologous PBMC composition is administered in a pulsatile manner. For example, the modified autologous PBMC composition may be administered intermittently over a period of time.

In addition, important embodiments of the invention include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

It is to be appreciated that the treatment of tumors or cancers with an effective amount of a modified autologous PBMC composition according to the present for a period of time sufficient to attenuate the fugetactic effect of the chemokine restores immune defenses against tumors, and may also allow anti-cancer agents (e.g., chemotherapeutic agents, radiotherapeutic agents, immunotherapy agents, and the like) to better access the tumor or cancer in order to reduce or eradicate the tumor or cancer. Without being bound by theory, it is believed that co-administration of the modified autologous PBMC compositions of the present invention and anti-cancer agents as described herein will lead to a synergistic response in a patient with a tumor or cancer, such that the patient has a better outcome than with either therapy alone. Anti-cancer agents include, without limitation, traditional cancer therapies, e.g. chemotherapy, radiotherapy, and/or vaccine therapy.

The modified autologous PBMC composition can be administered in combination with at least one anti-cancer therapy/agent. “In combination” refers to any combination, including sequential or simultaneous administration. In one embodiment, the anti-fugetactic agent is administered separately from the anti-cancer therapy/agent. In one embodiment, the anti-fugetactic agent is administered in a single composition with the anti-cancer agent(s).

The anti-cancer agent may be administered by any appropriate method. Dosage, treatment protocol, and routes of administration for anti-cancer agents, including chemotherapeutic agents, radiotherapeutic agents, immunotherapy agents, and anti-cancer vaccines, are known in the art and/or within the ability of a skilled clinician to determine, based on the type of treatment, type of cancer, etc.

In one aspect of the invention, the modified autologous PBMC composition and the anti-cancer agent(s) are administered sequentially. That is, the modified autologous PBMC composition is administered for a period of time sufficient to have an anti-fugetactic effect, and the anti-cancer agent is subsequently administered.

In one aspect of the invention, the anti-cancer agent is administered after the period of time of administration of modified autologous PBMC composition. In one embodiment, the anti-cancer agent is administered during a period of time wherein the fugetactic effect of the cancer cells/tumor is attenuated by the modified autologous PBMC composition. The length of time and modes of administration of the anti-cancer agent will vary, depending on the anti-cancer agent used, type of tumor being treated, condition of the patient, and the like. Determination of such parameters is within the capability of the skilled clinician.

In one embodiment, administration of the modified autologous PBMC composition and the anti-cancer agent is alternated. In a preferred embodiment, administration of the modified autologous PBMC composition and the anti-cancer agent is alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer.

In one embodiment, the modified autologous PBMC composition and/or anti-cancer agent is administered intravenously, subcutaneously, orally, or intraperitoneally. In a preferred embodiment, the modified autologous PBMC composition is administered proximal to (e.g., near or within the same body cavity as) the tumor. In one embodiment, the modified autologous PBMC composition is administered directly into the tumor or into a blood vessel feeding the tumor. In one embodiment, the modified autologous PBMC composition is administered systemically. In a further embodiment, the modified autologous PBMC composition is administered by microcatheter, or an implanted device, and an implanted dosage form.

In a preferred embodiment, the modified autologous PBMC composition and anti-cancer agent(s) are administered sequentially. For example, the modified autologous PBMC composition may be administered for a period of time sufficient to reduce or attenuate the fugetactic effect of the tumor, e.g. such that the modified autologous PBMC composition has an anti-fugetactic effect; the anti-cancer agent can then be administered for a period of time during which the fugetactic effect of the tumor is reduced or attenuated. In one embodiment, the modified autologous PBMC composition and anti-cancer agent are administered sequentially in an alternating manner at least until the condition of the patient improves.

Improvement of the condition of the patient includes, without limitation, reduction in tumor size, a reduction in at least one symptom of the cancer, elimination of the tumor and/or metastases thereof, increased survival of the patient, and the like.

In one embodiment, the modified autologous PBMC composition and/or the at least one additional anti-cancer agent are administered directly to the tumor site. In one embodiment, the modified autologous PBMC composition and/or the at least one additional anti-cancer agent are administered by direct injection into the tumor. In one embodiment, the modified autologous PBMC composition and/or the at least one additional anti-cancer agent are administered proximal to the tumor site. In a preferred embodiment, the modified autologous PBMC composition and/or the at least one additional anti-cancer agent are administered directly into a blood vessel associated with the tumor (e.g., via microcatheter injection into the blood vessels in, near, or feeding into the tumor).

### Chemotherapy Agents

In one aspect of the present invention, a modified autologous PBMC composition is administered in combination with a chemotherapy agent. The chemotherapy agent may be any agent having a therapeutic effect on one or more types of cancer. Many chemotherapy agents are currently known in the art. Types of chemotherapy drugs include, by way of non-limiting example, alkylating agents, antimetabolites, anti-tumor antibiotics, totpoisomerase inhibitors, mitotic inhibitors, corticosteroids, and the like.

Non-limiting examples of chemotherapy drugs include: nitrogen mustards, such as mechlorethamine (nitrogen mustard), chlorambucil, cyclophosphamide (Cytoxan®), ifosfamide, and melphalan); Nitrosoureas, such as streptozocin, carmustine (BCNU), and lomustine; alkyl sulfonates, such as busulfan; Triazines, such as dacarbazine (DTIC) and temozolomide (Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); Capecitabine (Xeloda®); Cytarabine (Ara-C®); Floxuridine; Fludarabine; Gemcitabine (Gemzar®); Hydroxyurea; Methotrexate; Pemetrexed (Alimta®); anthracyclines, such as Daunorubicin, Doxorubicin (Adriamycin®), Epirubicin, Idarubicin; Actinomycin-D; Bleomycin; Mitomycin-C; Mitoxantrone; Topotecan; Irinotecan (CPT-11); Etoposide (VP-16); Teniposide; Mitoxantrone; Taxanes: paclitaxel (Taxol®) and docetaxel (Taxotere®); Epothilones: ixabepilone (Ixempra®); Vinca alkaloids: vinblastine (Velban®), vincristine (Oncovin®), and vinorelbine (Navelbine®); Estramustine (Emcyt®); Prednisone; Methylprednisolone (Solumedrol®); Dexamethasone (Decadron); L-asparaginase; bortezomib (Velcade®). Additional chemotherapy agents are listed, for example, in U.S. Patent Application Pub. No. 2008/0300165, which is incorporated herein by reference in its entirety.

Doses and administration protocols for chemotherapy drugs are well-known in the art. The skilled clinician can readily determine the proper dosing regimen to be used, based on factors including the chemotherapy agent(s) administered, type of cancer being treated, stage of the cancer, age and condition of the patient, patient size, location of the tumor, and the like.

### Radiotherapy Agents

In one aspect of the present invention, a modified autologous PBMC composition is administered in combination with a radiotherapeutic agent. The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In one embodiment, the radiotherapeutic agent is delivered by a machine outside of the body (external-beam radiation therapy). In a preferred embodiment, the radiotherapeutic agent is placed in the body near the tumor/cancer cells (brachytherapy) or is a systemic radiation therapy.

External-beam radiation therapy may be administered by any means. Non-limiting examples of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent Non-limiting examples of internal radiation therapy include any radioactive agents that can be placed proximal to or within the tumor, such as Radium-226 (Ra-226), Cobalt-60 (Co-60), Cesium-137 (Cs-137), cesium-131, Iridium-192 (Ir-192), Gold-198 (Au-198), Iodine-125 (I-125), palladium-103, yttrium-90, etc. Such agents may be administered by seeds, needles, or any other route of administration, and my be temporary or permanent.

Systemic radiation therapy may be by any technique or agent. Non-limiting examples of systemic radiation therapy include radioactive iodine, ibritumomab tiuxetan (Zevalin®), tositumomab and iodine I 131 tositumomab (Bexxar®), samarium-153-lexidronam (Quadramet®), strontium-89 chloride (Metastron®), metaiodobenzylguanidine, lutetium-177, yttrium-90, strontium-89, and the like.

In one embodiment, a radiosensitizing agent is also administered to the patient Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like.

### Immunotherapy Agents

In one aspect of the present invention, a modified immune cell composition and/or unbound anti-fugetactic agent is administered in combination with an additional immunotherapy agent.

### Cellular Therapy

NK cells or T cells may be administered in combination with the compositions described herein. Generally, such T cells are modified and/or undergo adoptive cell transfer (ACT). ACT and variants thereof are well known in the art. See, for example, U.S. Pat. Nos. 8,383,099 and 8,034,334, which are incorporated herein by reference in their entireties.

U.S. Patent App. Pub. Nos. 2014/0065096 and 2012/0321666, incorporated herein by reference in their entireties, describe methods and compositions for T cell or NK cell treatment of cancer. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, each of which is incorporated herein by reference in its entirety.

In one embodiment, the NK cells or T cells used in the compositions and methods herein are autologous (i.e., derived from the patient). In one embodiment, the NK cells or T cells used in the compositions and methods herein are non-autologous (heterologous; e.g. from a donor or cell line). In one embodiment, the NK cells or T cells are a cell line derived from NK cells or T cell(s) or cancerous/transformed NK cells or T cell(s).

In one embodiment, the NK cell or T cell used in the methods and compositions described herein is genetically modified. In one embodiment, the cell is modified to express a CAR on the surface of the cell. In a preferred embodiment, the CAR is specific for the cancer being targeted by the method or composition. In one embodiment, the cell is modified to express a cell surface protein or cytokine. Non-limiting examples of modified T cells are described in U.S. Pat. No. 8,906,682; PCT Patent Pub. Nos. WO 2013154760 and WO 2014055668; each of which is incorporated herein by reference in its entirety.

Non-limiting examples of modified NK cells can be found, for example, in Glienke, et al. 2015, Advantages and applications of CAR-expressing natural killer cells, Frontiers in Pharmacol. 6, article 21; PCT Patent Pub. Nos. WO 2013154760 and WO 2014055668; each of which is incorporated herein by reference in its entirety.

In some embodiments, the NK cells are an NK cell line. NK cell lines include, without limitation, NK-92, NK-YS, KHYG-1, NKL, NKG, SNK-6, and IMC-1. See, Klingemann et al. Front Immunol. 2016; 7: 91, which is incorporated herein by reference in its entirety. Non-limiting examples of modified NK-92 cells are described, for example, in U.S. Pat. Nos. 7,618,817 and 8,034,332; and U.S. Patent Pub. Nos. 2002/0068044 and 2008/0247990, each of which is incorporated herein by reference in its entirety. Examples of modified NK-92 cells are available from ATCC as ATCC CRL-2408, ATCC CRL-2409, PTA-6670, PTA-6967, PTA-8837, and PTA-8836. Non-limiting examples of CAR-modified NK-92 cells can be found, for example, in Glienke, et al. 2015, Advantages and applications of CAR-expressing natural killer cells, Frontiers in Pharmacol. 6, article 21; which is incorporated herein by reference in its entirety.

In one embodiment, the T cell is a T cell line. Non-limiting examples of T cell lines include T-ALL cell lines, as described in U.S. Pat. No. 5,272,082, which is incorporated herein by reference in its entirety.

### Antibodies

Immunotherapy also refers to treatment with anti-tumor antibodies. That is, antibodies specific for a particular type of cancer (e.g., a cell surface protein expressed by the target cancer cells) can be administered to a patient having cancer. The antibodies may be monoclonal antibodies, polyclonal antibodies, chimeric antibodies, antibody fragments, human antibodies, humanized antibodies, or non-human antibodies (e.g. murine, goat, primate, etc.). The therapeutic antibody may be specific for any tumor-specific or tumor-associated antigen. See, e.g. Scott et al., Cancer Immunity 2012, 12:14, which is incorporated herein by reference in its entirety.

In one embodiment, the immunotherapy agent is an anti-cancer antibody. Non-limiting examples include trastuzumab (Herceptin®), bevacizumab (Avastin), cetuximab (Erbitux®), panitumumab (Vectibix®), ipilimumab (Yervoy®), rituximab (Rituxan), alemtuzumab (Campath®), ofatumumab (Arzerra®), gemtuzumab ozogamicin (Mylotarg®), brentuximab vedotin (Adcetris®), ⁹⁰Y-ibritumomab tiuxetan (Zevalin), and ¹³¹I-tositumomab (Bexxar®).

Additional antibodies are provided in Table 1.

TABLE 1
Anti-cancer antibodies

| | | | Indication first |
|---|---|---|---|
| Proprietary | | | approved or |
| name | Trade name | Target; Format | reviewed |
| Necitumumab | (Pending) | EGFR; Human | Non-small cell |
| | | IgG1 | lung cancer |
| Nivolumab | Opdivo | PD1; Human IgG4 | Melanoma |
| Dinutuximab | (Pending) | GD2; Chimeric | Neuroblastoma |
| | | IgG1 | |
| Blinatumomab | Blincyto | CD19, CD3; | Acute |
| | | Murine bispecific | lymphoblastic |
| | | tandem scFv | leukemia |
| Pembrolizumab | Keytruda | PD1; Humanized | Melanoma |
| | | IgG4 | |
| Ramucirumab | Cyramza | VEGFR2; Human | Gastric cancer |
| | | IgG1 | |
| Obinutuzumab | Gazyva | CD20; Humanized | Chronic |
| | | IgG1; | lymphocytic |
| | | Glycoengineered | leukemia |
| Ado-trastuzumab | Kadcyla | HER2; humanized | Breast cancer |
| emtansine | | IgG1; | |
| | | immunoconjugate | |
| Pertuzumab | Perjeta | HER2; humanized | Breast Cancer |
| | | IgG1 | |
| Brentuximab | Adcetris | CD30; Chimeric | Hodgkin |
| vedotin | | IgG1; | lymphoma, |
| | | immunoconjugate | systemic |
| | | | anaplastic large |
| | | | cell lymphoma |
| Ipilimumab | Yervoy | CTLA-4; Human | Metastatic |
| | | IgG1 | melanoma |
| Ofatumumab | Arzerra | CD20; Human | Chronic |
| | | IgG1 | lymphocytic |
| | | | leukemia |

### Immune Checkpoint Inhibitors

In one embodiment, the immunotherapy agent is a checkpoint inhibitor. Immune checkpoint proteins are made by some types of immune system cells, such as T cells, and some cancer cells. These proteins, which can prevent T cells from killing cancer cells, are targeted by checkpoint inhibitors. Checkpoint inhibitors increase the T cells' ability to kill the cancer cells. Examples of checkpoint proteins found on T cells or cancer cells include PD-1/PD-L1 and CTLA-4/B7-1/B7-2.

In one embodiment, the checkpoint inhibitor is an antibody to a checkpoint protein, e.g., PD-1, PDL-1, or CTLA-4. Checkpoint inhibitor antibodies include, without limitation, BMS-936559, MPDL3280A, MedI-4736, Lambrolizumab, Alemtuzumab, Atezolizumab, Ipilimumab, Nivolumab, Ofatumumab, Pembrolizumab, and Rituximab.

### Cytokines

In one embodiment, the immunotherapy agent is a cytokine. Cytokines stimulate the patient's immune response. Cytokines include interferons and interleukins. In one embodiment, the cytokine is interleukin-2. In one embodiment, the cytokine is interferon-alpha.

### Anti-Cancer Vaccines

In one aspect of the present invention, a modified autologous PBMC composition is administered in combination with an anti-cancer vaccine (also called cancer vaccine). Anti-cancer vaccines are vaccines that either treat existing cancer or prevent development of a cancer by stimulating an immune reaction to kill the cancer cells. In a preferred embodiment, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gp100 melanoma vaccine, and any other vaccine that stimulates an immune response to cancer cells when administered to a patient.

### Cancers

Cancers or tumors that can be treated with the modified autologous PBMC compositions and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In important embodiments, cancers or tumors escaping immune recognition include glioma, colon carcinoma, colorectal cancer, lymphoid cell-derived leukemia, choriocarcinoma, and melanoma.

In a preferred embodiment, the tumor is a solid tumor. In one embodiment, the tumor is a leukemia. In an especially preferred embodiment, the tumor over-expresses CXCL12. In one embodiment, tumor expression of CXCL12 can be evaluated prior to administration of a composition as described herein. For example, a patient having a tumor that is determined to express or over-express CXCL12 will be treated using a method and/or composition as described herein.

In one embodiment, the tumor is a brain tumor. It is contemplated that a brain tumor, e.g., an inoperable brain tumor, can be injected with a composition described herein. In one embodiment, an anti-fugetactic agent is administered directly to a brain tumor via a catheter into a blood vessel within or proximal to the brain tumor. Further discussion of catheter or microcatheter administration is described below.

### Pharmaceutical Compositions

The present invention also provides pharmaceutical compositions comprising an effective amount of the modified autologous PBMC compositions of the present invention and one or more pharmaceutically acceptable excipients. For preparing pharmaceutical compositions containing modified autologous PBMC compositions of the present invention, inert and pharmaceutically acceptable excipients or carriers are used. Liquid pharmaceutical compositions include, for example, solutions, suspensions, and emulsions suitable for intradermal, subcutaneous, parenteral, or intravenous administration. Sterile water solutions of the modified autologous PBMC compositions or sterile solutions of the modified autologous PBMC compositions in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

The pharmaceutical compositions containing modified autologous PBMC compositions can be administered for prophylactic and/or therapeutic treatments. In therapeutic applications, compositions are administered to a patient already suffering from a condition that may be exacerbated by the proliferation of tumor or cancer cells in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 1 mu.g to about 10 mg of the PAP peptide or fusion peptide biweekly for a 70 kg patient, with dosages of from about 50 mu.g to about 1 mg of the peptide biweekly for a 70 kg patient being more commonly used. The appropriate dose may be administered in weekly, biweekly, or monthly intervals. Single or multiple administrations of the compositions can be carried out with dose levels and pattern being selected by the treating physician. In any event, the pharmaceutical formulations should provide a quantity of the modified autologous PBMC compositions of this invention sufficient to provide the desired anti-fugetactic properties when administered to the patient, and to effectively inhibit tumor cell proliferation in the patient for therapeutic purposes.

Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990). The pharmaceutical compositions of the present invention can be administered by various routes, e.g., subcutaneous, intradermal, transdermal, intramuscular, intravenous, or intraperitoneal.

### Methods of Making Modified Immune Cells

In one aspect of this invention is provided a method for making modified immune cells as described herein.

In one embodiment, the method for making an immune cell composition comprises:

a) providing an immune cell composition;

b) incubating the immune cells with a fusion protein comprising a tumor antigen portion and an immune signaling factor portion for a period of time sufficient for the immune cells to become responsive to the tumor antigen; and

c) contacting the immune cells with an anti-fugetactic agent.

In one embodiment, the method for making an immune cell composition comprises:

a) providing an immune cell composition which is responsive to a tumor antigen; and

b) contacting the immune cells with an anti-fugetactic agent.

In one embodiment, the step of providing the immune cell composition includes removing immune cells from a patient having cancer.

In one embodiment, the immune cell composition had been made responsive to the tumor antigen (e.g., activated) by incubating the immune cells with a fusion protein comprising a tumor antigen portion and an immune signaling factor portion for a period of time sufficient for the immune cells to become responsive to the tumor antigen. In one embodiment, incubation occurs ex vivo/in vitro. In one embodiment, incubation occurred in the patient (in vivo) prior to extraction of the immune cells from the patient.

### Methods of Treatment

In one aspect of this invention is provided a method for treating cancer in a patient in need thereof by administration of a modified PBMC composition. In a preferred embodiment, the modified PBMC composition is administered in combination with at least one additional anti-cancer agent.

In one aspect, this invention relates to inhibition of metastasis of a tumor in a patient in need thereof by administration of a modified PBMC composition. Without being bound be theory, it is believed that the modified PBMC compositions as described herein can mobilize cancer cells out of niches where they are otherwise inaccessible to treatments and/or immune cells, and into the circulation where the cells can be targeted by anti-cancer agents and/or immune cells. Surprisingly, such mobilization does not lead to increased metastasis of the tumor, but rather decreases metastasis.

In one aspect, this invention relates to a method for killing a cancer cell expressing an amount of a chemokine sufficient to produce a fugetactic effect, which method comprises:

a) periodically contacting said cell with an effective amount of a modified autologous PBMC composition for a sufficient period of time so as to attenuate said fugetactic effect;

b) contacting said cell with at least one anti-cancer agent; and

c) optionally repeating a) and b) as necessary to kill said cell.

In one aspect, this invention relates to a method for treating a tumor in a mammal, said tumor expressing an amount of a chemokine sufficient to produce a fugetactic effect, which method comprises:

a) periodically administering to said mammal an effective amount of a modified autologous PBMC composition for a sufficient period of time so as to attenuate said fugetactic effect;

b) administering to said mammal at least one anti-cancer agent; and

c) optionally repeating a) and b) as necessary to provide an improvement in the condition of the mammal.

In one embodiment, the anti-cancer agent is administered after the period of time of administration of the modified immune cell composition. In one embodiment, the anti-cancer agent is administered during a period of time when the fugetactic effect is attenuated.

In one embodiment, the chemokine is CXCL12. In one embodiment, the cancer cell is a solid tumor cell. In one embodiment, the cancer cell is a leukemia cell. In one embodiment, the anti-cancer agent is administered within about 3 days of completion of contacting the cell with the anti-fugetactic agent. In one embodiment, the anti-cancer agent is administered within about 1 day of completion of contacting the cell with the anti-fugetactic agent.

In one aspect, this invention relates to a method for treating a solid tumor in a mammal which tumor expresses CXCL12 at a concentration sufficient to produce a fugetactic effect, the method comprising administering to said mammal an effective amount of modified immune cell composition for a sufficient period of time so as to inhibit said fugetactic effect, followed by administering to said mammal at least one anti-cancer agent. In one embodiment, the cancer cell is a solid tumor cell. In one embodiment, the cancer cell is a leukemia cell. In one embodiment, the anti-cancer agent is administered within about 3 days of completion of administration of the anti-fugetactic agent. In one embodiment, the anti-cancer agent is administered within about 1 day of completion of administration of the anti-fugetactic agent.

In one aspect, this invention relates to solid tumor cell expressing a chemokine, which cell has been contacted with a modified autologous PBMC composition and a chemotherapeutic agent. In one embodiment, the chemokine is CXCL12. In one embodiment, the cancer cell is a solid tumor cell. In one embodiment, the cancer cell is a leukemia cell.

In one aspect, this invention relates to a method to locally treat a solid tumor expressing CXCL12 at a concentration sufficient to produce a fugetactic effect in a patient, which method comprises:

a) identifying an artery or microartery feeding said tumor;

b) intra-arterially placing a catheter or microcatheter in said artery or microartery proximal to the flow of blood into said tumor wherein said catheter or microcatheter comprising a lumen for delivering a fluid there through and means for delivering said fluid;

c) periodically administering an effective amount of the modified immune cell composition through said catheter or said microcatheter to the artery or microartery feeding said tumor so as to inhibit said fugetactic effect fugetaxis induced by said tumor; and

d) subsequently administering an effective amount of the anti-cancer agent to the patient.

In one embodiment, the tumor is a brain tumor.

In one embodiment, the anti-cancer agent is administered using a catheter, a microcatheter, an external radiation source, or is injected or implanted proximal to or within the tumor. In one embodiment, the method further comprises repeating steps a, b, c, and/or d until the patient's condition improves. In one embodiment, the anti-cancer agent is a radiotherapeutic agent, such that the radiotherapeutic agent causes ablation of at least one blood vessel feeding said tumor.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: Determination of the Anti-Fugetactic Versus Fugetactic Amount of AMD3100

Freshly prepared and purified human CD3⁺ T cells were prepared from healthy donor peripheral blood. 20,000 T cells were loaded into the upper chamber of the Transwell in control, chemotactic or fugetactic settings with AMD3100 at concentrations between 0.1 μM and 10 μM. Migrated cells were counted in the lower chamber and migration quantitated as previously described. Vianello et al. The Journal of Immunology, 2006, 176: 2902-2914; Righi et al., Cancer Res.; 71(16); 5522-34, each of which is incorporated herein in its entirety.

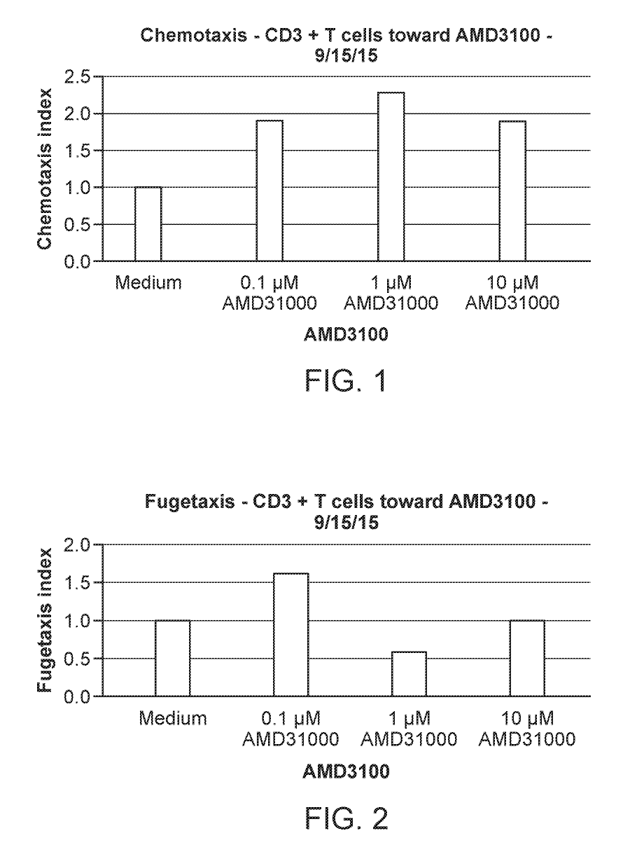

We saw clear evidence of binary or bimodal chemotactic (FIG. 1; CI 2.3 at 1 μM) and fugetactic (FIG. 2; CI=1.6 at 0.1 μM) responses of human CD3+ T cells to AMD3100 (where a CI or chemotactic index of 1.0 is the control). All wells were run in triplicate.

### Example 2: Determination of the Local Anti-Fugetactic Amount of AMD3100

For quantitative transmigration assays, purified human CD3⁺ T cells (approximately 2×10⁴ cells) are added to the upper chamber of a Transwell® insert in each well, to a total volume of 150 μl of Iscove's modified medium. Tumor cells isolated from a mammalian tumor in DMEM containing 0.5% FCS, are added in the lower, upper, or both lower and upper chambers of the Transwell to generate a standard “checkerboard” analysis of cell migration, including measurements of chemotaxis, fugetaxis, and chemokinesis.

To determine the anti-fugetactic concentration of AMD3100, the T cells are incubated with 0.01 μM to 10 mM AMD3100 prior to addition to the chamber.

Cells are harvested from the lower chamber after 3 h, and cell counts are performed using a hemocytometer.

It is expected that T cells that are pre-incubated with a concentration of AMD3100 will exhibit a bimodal effect, with anti-fugetactic effects observed at lower concentrations and fugetactic effects at higher concentrations.

### Example 3: Treatment of Prostate Cancer with Sipuleucel-T and an Fugetactic Agent

Antigen presenting cells (APC) are isolated from a 65 year old patient with prostate cancer, exposed to PAP antigen and matured with GM-CSF. The APC are administered to the patient. After a period of time, the APC stimulate a specific T-cell response against PAP antigen. When the T-cell response is detected, a population of PBMCs are obtained from the patient's blood, mixed and incubated with AMD3100. The patient receives 1.6×10⁷ modified cells/AMD3100 composition via direct infusion into the tumor. Alternatively, the cells and AMD310 can be administered separately and substantially simultaneously. It is contemplated that treatment with the modified cells and AMD3100 will have a synergistic is effect, such that the co-treatment results in decrease prostate cancer progression.

1. An ex vivo immune cell composition comprising immune cells that are responsive to a tumor antigen, and an anti-fugetactic agent, wherein said modified immune cell composition has anti-fugetactic properties for the treatment of a tumor or cancer in a patient.

2. The immune cell composition of claim 1, wherein the immune cells are derived from the patient.

3. The immune cell composition of claim 1, wherein the anti-fugetactic agent is associated with at least one receptor on the cell surface.

4. The immune cell composition of claim 3, further comprising anti-fugetactic agent that is not associated with the receptor.

5. The immune cell composition of claim 3, wherein the receptor is CXCR4.

6. The immune cell composition of claim 1, wherein said anti-fugetactic agent is selected from the group consisting of AMD3100 or derivative thereof, KRH-1636, T-20, T-22, T-140, TE-14011, T-14012, TN14003, TAK-779, AK602, SCH-351125, Tannic acid, NSC 651016, thalidomide, GF 109230X, an antibody that interferes with dimerization of a fugetactic chemokine, and an antibody that interferes with dimerization of a receptor for a fugetactic chemokine.

7. The immune cell composition of claim 6, wherein said anti-fugetactic agent is AMD3100.

8. The immune cell composition of claim 1, wherein said immune cells are PBMCs.

9. (canceled)

10. The immune cell composition of claim 1, wherein the immune cells from a patient that are responsive to a tumor antigen are obtained from the patient after treatment with a vaccine or antigen presenting cell that induces an immune response against the tumor antigen.

11. The immune cell composition of claim 1, wherein the immune cells were activated by incubation with a fusion protein.

12. The immune cell composition of claim 11, wherein the fusion protein comprises a tumor antigen portion and an immune signaling factor portion.

13. (canceled)

14. A pharmaceutical composition comprising an effective amount of the ex vivo immune cell composition of claim 1 and one or more pharmaceutically acceptable excipients.

15. The pharmaceutical composition of claim 14, wherein said anti-fugetactic agent is associated with one or more receptors on the surface of the immune cells.

16. The pharmaceutical composition of claim 15, wherein the one or more receptors comprise CXCR4.

17. The pharmaceutical composition of claim 15, further comprising anti-fugetactic agent that is not associated with the receptor.

18. The pharmaceutical composition of claim 14, wherein said anti-fugetactic agent is selected from the group consisting of AMD3100 or derivative thereof, KRH-1636, T-20, T-22, T-140, TE-1401 1, T-14012, TN14003, TAK-779, AK602, SCH-351125, Tannic acid, NSC 651016, thalidomide, GF 109230X, an antibody that interferes with dimerization of a fugetactic chemokine, and an antibody that interferes with dimerization of a receptor for a fugetactic chemokine.

19. The pharmaceutical composition of claim 18, wherein said anti-fugetactic agent is AMD3100.

20. The pharmaceutical composition of claim 14, wherein said immune cells are PBMCs.

21-22. (canceled)

23. The pharmaceutical composition of claim 14, wherein the immune cells were activated by incubation with a fusion protein.

24. The pharmaceutical composition of claim 14, wherein the fusion protein comprises a tumor antigen portion and an immune signaling factor portion.

25-29. (canceled)

30. A method of treating cancer in a patient, comprising administration of the cell composition of claim 1.

31. A method of treating cancer in a patient in need thereof, comprising:
a) providing immune cells derived from the patient;
b) incubating the immune cells with a fusion protein comprising a tumor antigen portion and an immune signaling factor portion for a period of time sufficient for the immune cells to become responsive to the tumor antigen;
c) contacting the immune cells with an anti-fugetactic agent; and
d) administering said immune cells to the patient.

32-33. (canceled)

34. The method of claim 31, wherein the immune cells are PBMCs.

35. The method of claim 31, wherein said anti-fugetactic agent is selected from the group consisting of AMD3100 or derivative thereof, KRH-1636, T-20, T-22, T-140, TE-14011, T-14012, TN14003, TAK-779, AK602, SCH-351 125, Tannic acid, NSC 651016, thalidomide, OF 10923 OX, an antibody that interferes with dimerization of a fugetactic chemokine, and an antibody that interferes with dimerization of a receptor for a fugetactic chemokine.

36. The method of claim 35, wherein the antifugetactic agent is AMD3100.

37. A method for making an immune cell composition, the method comprising:
a) providing an immune cell composition;
b) incubating the immune cells with a fusion protein comprising a tumor antigen portion and an immune signaling factor portion for a period of time sufficient for the immune cells to become responsive to the tumor antigen; and
c) contacting the immune cells with an anti-fugetactic agent.

38-39. (canceled)

40. The method of claim 37, wherein the immune cells are PBMCs.

41. The method of claim 37, wherein said anti-fugetactic agent is selected from the group consisting of AMD3100 or derivative thereof, KRH-1636, T-20, T-22, T-140, TE-14011, T-14012, TN14003, TAK-779, AK602, SCH-351 125, Tannic acid, NSC 651016, thalidomide, GF 10923 OX, an antibody that interferes with dimerization of a fugetactic chemokine, and an antibody that interferes with dimerization of a receptor for a fugetactic chemokine.

42. The method of claim 41, wherein the antifugetactic agent is AMD3100.

43. (canceled)
